(19) United States
(12) Patent Application Publication
(10) Pub. No.: US 2007/0185065 A1
(43) Pub. Date: Aug. 9, 2007

(54) Combination therapy for coronary artery disease

(76) Inventor: Vikramjit Chhokar, Pewaukee, WI (US)

Correspondence Address: BOYLE FREDRICKSON NEWHOLM STEIN & GRATZ, S.C., 250 E. WISCONSIN AVENUE, SUITE 1030, MILWAUKEE, WI, 53202, US

(21) Appl. No.: 11/346,703

(22) Filed: Feb. 3, 2006

(51) Int. Cl. A61K 31/60; A61K 31/58; A61K 31/519; A61K 31/401; A61K 31/4743; A61K 31/366; A61K 31/22

(52) U.S. Cl. 514/160; 514/651; 514/356; 514/171; 514/176; 514/381; 514/394; 514/571; 514/262.1; 514/423; 514/460; 514/548; 514/301; 514/409

(57) ABSTRACT

A combination therapy and methods for administering the combination therapy is provided that reduces mortality or morbidity of coronary artery disease (CAD) or coronary artery disease equivalents in individuals. The combination therapy can be administered to an individual in various forms, and the therapy is a medicament combining an anti-platelet agent(s), a beta-blocker, renin-angiotensin-aldosterone system (RAAS) inhibitor(s), and/or cholesterol modifying agent(s).

### FIELD OF THE INVENTION

The present invention relates to coronary artery disease (CAD) medications in general, and in particular to a combination medication therapy including an Antiplatelet agent(s), a Beta blocker, a Renin-Angiotensin-Aldosterone System (RAAS) inhibitor(s) and a cholesterol modifying agent(s) for reducing the mortality or morbidity of coronary artery disease or coronary artery disease equivalents, and a method of administering the medication therapy to a patient in need of such therapy.

### BACKGROUND OF THE INVENTION

Survivors of acute myocardial infarctions (MI) have a significant risk of future cardiovascular events. A number of modalities have proven to be beneficial in the management of patients with coronary artery disease (CAD) or CAD equivalent, in terms of reducing death (mortality) or complication of the underlying disease (morbidity). Equivalents of coronary artery disease are other pathologies that have been considered to require management as if treatment were for secondary prevention of CAD itself. CAD equivalents have been defined by Adult Treatment Panel (ATP) III and include the following conditions:

Diabetes mellitus (Type I or Type II)

Symptomatic carotid artery disease

Peripheral arterial disease

Abdominal aortic aneurysm

Multiple risk factors that confer a 10-year risk of CAD >20 percent

Treatments of CAD are based on patient characteristics and generally include the administration of antiplatelet agents, angiotensin converting enzyme (ACE) inhibitors or angiotensin II receptor blockers (ARBs), aldosterone antagonists, beta blockers, cholesterol modifying agents, among other treatment therapies.

Of particular interest to the patent, antiplatelet agents (such as aspirin, clopidogrel), beta blockers, RAAS inhibitor(s), (such as ACE Inhibitors, ARBs, or aldoterone receptor blockers) and cholesterol modifying agents (such as HMG-CoA reductase inhibitor gemfibrozil or niacin), have all been separately validated in reducing mortality or morbidity of individuals who have coronary artery disease or coronary artery disease equivalents. A multitude of trials have been completed that address the benefits of these different individual classes of agents.

While each of the aforementioned medications/classes provides some benefit with regard to individuals having these types of histories/risk factors, a significant drawback to these medications is their administration. More particularly, each medication is administered separately, resulting in a large volume or number of pills or other dosage forms that must be taken by an individual in order to provide the appropriate benefit/treatment to the individual. Currently, these medications are administered individually, such that the benefit provided by the single medication may not be effectively utilized in conjunction with other medications that could provide additional and/or synergistic benefits to the individual.

Moreover, studies have shown that as the number of medications prescribed increases, patient compliance with taking those medications decreases. By providing a simplified dose form with specific classes of medication, there would be the potential benefit of improved compliance as well as medication synergy. Thus, this invention should result in decreased mortality and morbidity through improved patient compliance and combination medication benefits.

Combination medications including inhibitors of the renin-angiotensin system have been developed previously. Specifically, in Scholkens et al. U.S. Patent Application Publication No. US 2005/0101658, an inhibitor of the renin-angiotensin system or a pharmaceutically acceptable derivative thereof is optionally utilized in conjunction with another compound for the treatment of cardiovascular events, namely, the prevention or reduction of the risk of onset of diabetes. However, this publication focuses solely on the utility of the inhibitors of the renin-angiotensin system in the treatment of and prevention of diabetes. For those individuals undergoing treatment for ongoing coronary artery disease, such preventative measures are not effective as the disease is already present.

As a result, it is desirable to develop a medication therapy and method of administering the therapy which would be formed by combination of these agents that are beneficial and/or synergistic in patients with CAD or an equivalent thereof. Such a medication therapy would provide a combination of the benefits of all of the therapeutic agents in a single pill or other dosage form(s), while also significantly reducing the problems of administration and possibly improving patient compliance compared administration of these agents separately.

### BRIEF SUMMARY OF THE INVENTION

According to a primary aspect of the present invention, a medication therapy and method of use or administration of the medication therapy are provided which enable an individual having a history of coronary artery disease (CAD) or a CAD equivalent to take the medication therapy which includes a number of different medications in the single therapy. Medications which are included in this therapy include anti-platelet agents, beta blockers, renin-angiotensin-aldosterone system (RAAS) inhibitors, and cholesterol modifying agents. Each of these medications by themselves has proven benefits in preventing occurrence of coronary artery disease or equivalents thereof in individuals. However, in the therapy form of the present invention and method for use of this therapy form, each of these medications is combined into the single therapy, e.g., a single pill, such that the therapy can be easily and continually administered to an individual in order to easily provide the benefits of each of the medications contained within therapy. Furthermore, while each class of medication provides some benefit, trials have shown that combination of each class of medication together provide additional benefit. Thus, a patient taking all four classes of medication would be better protected then a patient taking just one class of medication. The therapy and method of administration of the therapy of the present invention would reduce cardiovascular events such as stroke, congestive heart failure, cardiovascular death, myocardial infarction, worsening of angina, and possible cardiac arrest. Additionally the need for revascularization procedures, such as coronary artery bypass graft surgery (CABG), Percutaneous Coronary Intervention (PCI), Peripheral Angioplasty Surgery, Amputation, Carotid Endarterectomy may be prevented. Finally, diabetic complications such as overt nephropathy, renal dialysis, or microalbuminuria can be prevented in a broad population of high-risk patients, by use of the combination of the medication therapy proposed in this art.

According to another aspect of the present invention, a method for treating individuals with histories of coronary artery disease or equivalent thereof is provided which involves administering the medication therapy including a number of components proven effective in the treatment of coronary artery disease, namely an effective amount of an anti-platelet agent(s), a beta blocker, RAAS inhibitor(s) and/or cholesterol modifying agent(s) in order to prevent or reduce mortality or morbidity of individuals with coronary artery disease or a coronary artery disease equivalent.

According to a further aspect of the present invention, a new combination medication dosage form and corresponding method of administration is provided to prevent or reduce mortality or morbidity individuals with coronary artery disease or coronary artery disease equivalent though administering to a patient in need of such prevention an effective amount of an anti-platelet agent(s), a beta blocker, RAAS inhibitor(s) and/or cholesterol modifying agent(s) in the new dosage form.

Numerous other aspects, features, and advantages of the present invention will be made apparent in the following detailed description.

### DETAILED DESCRIPTION OF THE INVENTION

The present invention is a new combination medication therapy and method of administration of the therapy to prevent cardiovascular events that includes an effective amount of anti-platelet agent(s), beta blocker, RAAS inhibitor(s) or cholesterol modifying agent(s), optionally together with one or more other optional ingredients.

The following definitions are used for the following terms and phrases throughout the present application:

The phrase “combination therapy”, in defining use of anti-platelet agent(s), beta blocker, inhibitor(s) of the renin angiotensin-aldosterone system (RAAS) and cholesterol modifying agent(s), among others, embraces the administration of each agent in a sequential manner in a regimen that will provide beneficial effects of the drug combination, and embraces co-administration of these agents in a substantially simultaneous manner, such as by oral ingestion of a single capsule/pill having a fixed ratio of these active agents or ingestion of multiple, separate capsules/pill for each agent. “Combination therapy” also includes simultaneous or sequential administration by intravenous, intramuscular or other parenteral routes into the body, including direct absorption through mucous membrane tissues, as found in the sinus passages. Sequential administration also includes drug combination where the individual elements may be administered at different times and/or by different routes but which act in combination to provide a beneficial effect.

The phrase “effective amount” is intended to qualify the amount of each agent for use in the combination therapy which will achieve the goal of preventing cardiac events while avoiding adverse side effects typically associated with each agent.

“High risk” patients are, for instance, those patients who are at risk having a cardiovascular event due to a manifest coronary heart disease, a history of transient ischemic attacks or stroke, diabetes or a history of peripheral vascular disease.

The phrase “inhibitor of the renin-angiotensin aldosterone system (RAAS) or a pharmaceutically acceptable derivative thereof” as used herein includes any compound which by itself or upon administration blocks the negative effects of angiotensin II or aldosterone on the vasculature either by reducing the synthesis of or blocking the effect at the receptor. Inhibitors of the RAAS include angiotensin enzyme (ACE) inhibitors, angiotensin II receptor blockers (ARBs), and aldosterone blockers, and the pharmaceutically acceptable derivatives thereof including prodrugs and metabolites.

The phrase “coronary artery disease” (CAD) is used here in to denote a condition (as sclerosis or thrombosis) that reduces the blood flow through the coronary arteries to the heart muscle.

The phrase “coronary artery disease equivalent” (CAD equivalent) is used herein to denote Diabetes mellitus (Type I or Type II), Symptomatic carotid artery disease, Peripheral arterial disease, Abdominal aortic aneurysm or Multiple risk factors that confer a 10-year risk of CAD >20 percent.

The word “mortality” is used to denote the proportion of deaths to population (death rate).

The word “morbidity” is used to describe a diseased state or symptom or the incidence of disease i.e. the rate of sickness.

The word “statins” is used herein to describe competitive inhibitors of 3-hydroxy-3-methylglutaryl-coenzyme A (HMG-CoA) reductase an enzyme that catalyzes the rate-limiting step in cholesterol biosynthesis.

The phrase “active ingredient” or active compound as used herein to describe the components of agents used to form the combination therapy of the present invention including anti-platelet agents, beta-blockers, RAAS inhibitors and cholesterol modifying agents. These types of ingredients are utilized in combination with one another to form the combination therapy that is the subject of the present invention. It has been found that the combination of each of these beneficial medications provides synergistic improved benefits to the therapy when the medications are combined to form the combination therapy of the present invention with regard to the treatment of coronary artery disease or an equivalent thereof.

The following discussion relates to the individual components of the combination therapy and their forms and benefits for the combination therapy.

I. Antiplatelet Agent

A number of anti-platelet agents have been shown to provide benefit in individual with CAD or CAD equivalent. Among these are: aspirin, dipyridamole, clopidogrel, ticlopidine, and glycoprotein IIb/IIIa inhibitors.

The mechanism of action varies with each agent. For example, aspirin works by blocking the enzyme cyclooxygenase (prostaglandin G/H synthase) that mediates the first step in the biosynthesis of prostaglandins and thromboxanes (including TXA2) from arachidonic acid. Dipyridamole inhibits phosphodiesterase-mediated breakdown of cyclic AMP, which prevents platelet activation by multiple mechanisms. The thienopyridines clopidogrel and ticlopidine achieve their antiplatelet effect by blocking the binding of ADP (Adenosine Diphospate) to a specific platelet receptor, thereby inhibiting the activation of the GP IIb/IIIa complex and platelet aggregation. Finally, Glycoprotein IIb/IIIa antibodies and receptor antagonists inhibit the final common pathway of platelet aggregation (the cross-bridging of platelets by fibrinogen binding to the GP IIb/IIIa receptor) and may also prevent initial adhesion to the vessel wall.

Each of these agents has been shown to provide benefit in treating of CAD or an equivalent thereof. Aspirin is the most extensively studied agent. Aspirin has been shown to provide secondary prevention of cardiovascular disease (CVD) after acute myocardial infarction (MI), occlusive stroke, transient ischemic attack (TIA), stable angina, and coronary artery bypass to reduce risks of MI, stroke and vascular death. Additionally, aspirin has shown efficacy in acute ischemic syndromes such as acute MI and unstable angina (UA). Furthermore, aspirin is considered a primary protection of initial MI. Finally, and of particular interest, aspirin reduces the risks of subsequent MI, stroke and vascular death and other morbidities of CAD.

The mechanism for the benefit of aspirin in CVD relates to its ability to irreversibly inhibit platelet dependent cyclooxygenase, which decreases aggregability of the platelet, thereby reducing the risk of thrombotic vascular events. Additional benefits of aspirin may be conferred by modification of certain proinflammatory cytokines and C-reactive protein (CRP); as in a randomized, double blind, placebo controlled trial, six weeks of aspirin therapy significantly reduced these markers of inflammation.

Secondary prevention with long-term aspirin therapy has also been validated. Aspirin has been shown to reduce the risk of subsequent myocardial infarction (MI), stroke, and vascular death among patients with a wide range of prior manifestations of cardiovascular disease. The magnitude and range of benefit was conclusively demonstrated in the Antithrombotic Trialists' Collaboration overview, which analyzed the results of 195 randomized trials of antiplatelet therapy, principally with aspirin, among more than 135,000 high-risk patients with prior evidence of CVD, including prior or acute MI, prior or acute stroke or transient ischemia attacks (TIA), and other high-risk groups such as UA, stable angina, peripheral vascular disease, CABG, PCI, atrial fibrillation, and valvular disease.

Although aspirin is the most studied antiplatelet agent, other antiplatelet agents, such as clopidogrel, have been shown to have similar efficacy. Clopidogrel mechanism of action is via blockade of the ADP receptors, which prevent fibrinogen binding at that site and thereby reduce the possibility of platelet adhesion and aggregation. In the PCI-CURE, CREDO, and CURE trials of patients with non-ST elevation acute coronary syndrome (ACS), clopidogrel was shown to provide benefit over the time course studied from nine to twelve months. Moreover, the benefit of clopidogrel for longer than one year was seen in the CAPRIE trial. This study compared the use of clopidogrel to the use of aspirin in patients with atherosclerosis not limited to MI. Clopidogrel therapy was associated with a significant reduction in fatal or nonfatal MI, and the magnitude of this benefit increased progressively over the three year period of the study.

In summation, anti-platelet agents clearly provide benefit and prevent mortality and morbidity in individuals with CAD or CAD equivalent.

II. Beta Blockers

Another class of medication that has been shown to reduce mortality and morbidity in CAD patients are beta-blockers. Beta-blockers have been shown to improved survival in patients with CAD, and in particular, in post myocardial infarction, patients undergoing coronary artery bypass graft surgery and patients with left ventricular systolic dysfunction and heart failure.

The mechanism of action is through inhibition of beta-adrenergic receptors. Beta-blockers can be selective to beta 1-adrenergic receptors or nonselective with interaction of both B1 and B2 receptors. Some nonselective beta-blockers have vasodilating properties as a result of selective alpha-1 antagonism, such as Carvedilol. The beneficial therapeutic effect of beta blockade in ischemic patients is mediated by a reduction in myocardial oxygen demand. Myocardial oxygen demand varies directly with heart rate, contractility, and left ventricular wall stress, each of which is diminished by beta blockade. The decrease in wall stress is mediated in part by the antihypertensive action of these drugs.

The efficacy of the beta-blocker metoprolol was demonstrated in the International Multicenter Angina Exercise (IMAGE) study in which 280 patients with chronic stable angina were randomized to six weeks therapy with long-acting preparations of metoprolol (200 mg daily) or nifedipine (20 mg BID). Metoprolol reduced the frequency of angina and increased the mean exercise time to 1-mm ST segment depression. Furthermore, the increase in exercise time was greater than that seen with nifedipine (70 versus 43 seconds, p<0.05).

In addition to the benefits in individuals with angina, beta-blockers have been shown to reduce morbidity and mortality post myocardial infarction, in multiple trials. In a systematic review, the overall mortality in 31 long-term trials that included almost 25,000 patients was 10.1 percent, and beta-blockers reduced the odds of death by 23 percent (95% CI 15-31 percent). Most patients are continued on an oral beta-blocker indefinitely. A meta-analysis of 54,234 patients entered into 82 randomized trials found that short-term beta blockade immediately after an acute MI was unlikely to be of major benefit unless treatment was continued long-term. Of particular interest, the continuing benefit of chronic beta-blocker therapy has been firmly established in a number of major trials for high-risk patients. In a review from the beta-blocker in Heart Attack Trial (BHAT), survivors at 12 months who were at high-risk and were treated with beta-blockers had a 43 percent reduction in the subsequent risk for death. High-risk patients treated as late as three to eight years after an MI still appear to benefit from beta-blocker therapy, although survival may not be improved in patients treated with an implantable cardioverter-defibrillator. Thus, beta-blockers confer survival benefit and reduce future adverse effect in individuals with underlying CAD or CAD equivalent.

Useful beta blockers include, but are not limited to, Betapace (sotalol), Blocadren (timolol), Brevibloc (esmolol), Cartrol (carteolol), Coreg (carvedilol), Corgard (nadolol), Inderal (propranolol), Inderal-LA (propranolol), Kerlone (betaxolol), Levatol (penbutolol), Lopressor (metoprolol), Normodyne (labetalol), Sectral (acebutolol), Tenormin (atenolol), Toprol-XL (metoprolol), Trandate (labetalol), Visken (pindolol), Zebeta (bisoprolol).

III. RAAS Inhibitors

The renin-angiotensin-aldosterone system (RAAS) can be interfered with by inhibition of the enzymes synthesizing angiotensins or by blocking the corresponding receptors at the effector sites or with specific aldosterone blocking agents. Collectively, inhibitions of RAAS have been shown to provide benefit in individuals with CAD or CAD equivalent.

The mechanism of action for ACE inhibitors is via an inhibition of angiotensin-converting enzyme (ACE) that prevents conversion of angiotensin I to angiotensin II, a potent vasoconstrictor, resulting in lower levels of angiotensin II, which causes an consequent increase in plasma renin activity and a reduction in aldosterone secretion. Angiotensin Receptor Blockers (ARBs) work as their name implies by directly blocking angiotensin II receptors and thus preventing the action of angiotensin II. Aldosterone blockers act by binding at mineralocorticoid receptors and thereby preventing the action of aldosterone.

The subset of the RAAS inhibitors suitable for use in the medication therapy and method of the present invention termed “angiotensin converting enzyme inhibitor” (“ACE inhibitor”) is intended to embrace any agent or compound, or a combination of two or more agents or compounds, having the ability to block, partially or completely, the rapid enzymatic conversion of the physiologically inactive decapeptide form of angiotensin (“Angiotensin I”) to the vasoconstrictive octapeptide form of angiotensin (“Angiotensin II”).

Examples of ACE inhibitors suitable for use herein are, but not limited to, the following compounds: AB-103, ancovenin, benazeprilat, BRL-36378, BW-A575C, CGS-13928C, CL242817, CV-5975, Equaten, EU4865, EU-4867, EU-5476, foroxymithine, FPL 66564, FR-900456, Hoe-065, 15B2, indolapril, ketomethylureas, KR1-1177, KR1-1230, L681176, libenzapril, MCD, MDL-27088, MDL-27467A, moveltipril, MS41, nicotianamine, pentopril, phenacein, pivopril, rentiapril, RG-5975, RG-6134, RG-6207, RGH0399, ROO-911, RS-10085-197, RS-2039, RS 5139, RS 86127, RU-44403, S-8308, SA-291, spiraprilat, SQ26900, SQ-28084, SQ-28370, SQ-28940, SQ-31440, Synecor, utibapril, WF-10129, Wy-44221, Wy-44655, Y-23785, Yissum, P-0154, zabicipril, Asahi Brewery AB-47, alatriopril, BMS 182657, Asahi Chemical C-111, Asahi Chemical C-112, Dainippon DU-1777, mixanpril, Prentyl, zofenoprilat, I (−(1-carboxy-6-(4-piperidinyl)hexyl)amino)-1-oxo-propyl octahydro-1H-indole-2-carboxylic acid, Bioproject BP1.137, Chiesi CHF 1514, Fisons FPL-66564, idrapril, perindoprilat and Servier S-5590, alacepril, benazepril, captopril, cilazapril, delapril, enalapril, enalaprilat, fosinopril, fosinoprilat, imidapril, lisinopril, perindopril, quinapril, ramipril, ramiprilat, saralasin acetate, temocapril, tran olapril, trandolaprilat, ceranapril, moexipril, quinaprilat and spirapril.

The phrase “ACE inhibitor” also embraces so-called NEP/ACE inhibitors (also referred to as selective or dual acting neutral endopeptidase inhibitors) which possess neutral endopeptidase (NEP) inhibitory activity and angiotensin converting enzyme (ACE) inhibitory activity. Examples of NEP/ACE inhibitors particularly preferred and suitable for use herein are those disclosed in U.S. Pat. Nos. 5,508,272, 5,362,727, 5,366,973, 5,225,401, 4,722,810, 5,223,516, 5,552,397, 4,749,688, 5,504,080, 5,612,359, 5,525,723, 5,430,145, and 5,679,671, and European Patent Applications 0481522, 0534263, 0534396, 0534492, and 0671172, each of which is incorporated expressly herein for reference. Especially preferred is the NEP/ACE inhibitor omapatrilat (disclosed in U.S. Pat. No. 5,508,272) or MDL100240 (disclosed in U.S. Pat. No. 5,430,145).

Another subset of the RAAS inhibitors that are suitable for use in medication therapy and method of the present invention termed “angiotensin II antagonist” is intended to embrace any agent or compound, or a combination of two or more agents or compounds, having the ability to block, partially or completely the binding of angiotensin II at angiotensin receptors, specifically at the AT.sub.1 receptor. These agents are also known as Angiotension Receptor Blockers (ARBs). Examples of angiotensin II antagonists suitable for but not limited for use herein are, for instance, the following compounds: Saralasin acetate, candesartan cilexetil, CGP-63170, EMD-66397, KT3-671, LR-B/081, valsartan, A-81282, BIBR-363, BIBS-222, BMS-184698, candesartan, CV-11194, EXP-3174, KW-3433, L-161177, L-162154, LR-B/057, LY-235656, PD-150304, U-96849, U-97018, UP-275-22, WAY-126227, WK-1492.2K, YM-31472, losartan potassium, E-4177, EMD-73495, eprosartan, HN-65021, irbesartan, L-159282, ME-3221, SL-91.0102, Tasosartan, Telmisartan, UP-269-6, YM-358, CGP-49870, GA-0056, L-159689, L-162234, L-162441, L-163007, PD-123177, A-81988, BMS-180560, CGP-38560A, CGP48369, DA-2079, DE-3489, DuP-167, EXP-063, EXP-6155, EXP-6803, EXP-7711, EXP-9270, FK-739, HR-720, ICI-D6888, ICI-D7155, ICI-D8731, isoteoline, KR1-1177, L-158809, L-158978, L-159874, LR B087, LY-285434, LY-302289, LY-315995, RG-13647, RWJ-38970, RWJ-46458, S-8307, S-8308, saprisartan, sara lasin, Sarmesin, WK-1360, X-6803, ZD-6888, ZD-7155, ZD-8731, BIBS39, C1-996, DMP-811, DuP-532, EXP-929, L-163017, LY-301875, XH-148, XR-510, zolasartan and PD-123319. A group of angiotensin II antagonists of particular interest are saralasin acetate, candesartan cilexetil, valsartan, candesartan, losartan potassium, eprosartan, irbesartan, tasosartan, or telmisartan. Examples of renin inhibitors suitable for use herein are, for instance, the following compounds: enalkrein; RO 42-5892; A 65317; CP 80794; ES 1005; ES 8891; SQ 34017; CGP 29287; CGP 38560; SR 43845; U-71038; A 62198; A 64662, A-69729, FK 906 and FK 744.

The final RAAS inhibitor is aldosterone blockers of note the only one under patent is eplerenone U.S. Pat. Nos. 6,410,054; 6,534,093; 6,558,707 and 6,863,902. Eplerenone or sprinolactone may be used in this combination therapy.

Many of these ACE inhibitors are commercially available, For example, ramipril (known from EP 79022) is sold by Aventis, e.g. under the trademark Delix® or Altace®. Enalapril or Enalapril Maleate, and Lisinopril are sold by Merck & Co. Enalapril is sold under the trademark Vasotec®. Lisinpril is sold under the trademark Prinivil®. Some of these agents are also available in generic forms and made by multiple manufacturers.

There are a number of trials showing the efficacy of ACE inhibitors. Most notably the SAVE trial, in which 2231 asymptomatic patients with an LVEF 40 percent were randomly assigned to treatment with either captopril (12.5 mg TID increasing to a final target dose of 50 mg TID) or placebo starting 3 to 16 days after MI. At a mean follow-up of 42 months, captopril therapy was associated with a 19 percent (95% CI 3 to 32 percent) decrease in mortality (20 versus 25 percent for placebo), a 37 percent reduction in the incidence of severe HF, a 22 percent reduction in hospitalization for HF, and a 25 percent reduction in the incidence of recurrent MI.

Furthermore, the efficacy of ACE inhibition in patients who develop overt HF post-MI was evaluated in the AIRE trial, which randomly assigned 2006 patients with clinical evidence of HF to ramipril or placebo between day 3 and 10 post-MI. After an average of 15 months follow-up, ramipril was associated with a 27 percent (95% CI 11 to 40 percent) decrease in mortality (17 percent versus 23 percent with placebo). Other benefits included a 23 percent reduction in the incidence of severe, resistant HF by 23 percent (14 versus 18 percent for placebo) and a 30 percent reduction in sudden death.

The survival benefit was maintained long-term. Among 603 patients from the United Kingdom entered into AIRE who were followed for a mean of 59 months, the mortality was lower in patients originally randomized to ramipril compared to placebo (28 versus 39 percent), representing a relative risk reduction of 36 percent (95% CI 15 to 52 percent).

A meta-analysis of three trials (SAVE, AIRE, and TRACE), all of which were included in the late administration group, documented both a long-term mortality benefit and additional improvements in outcome with ACE inhibition.

Two major trials have compared an ARB to an ACE inhibitors in patients who have had an acute MI: OPTIMAAL and VALIANT. Based upon VALIANT, valsartan at a dose of 160 mg twice daily appears to be as effective as an ACE inhibitor for reduction in mortality after an MI. It is possible that losartan at a higher dose than that used in OPTIMAAL (eg, 100 mg once daily) would be similarly effective, although this has not been proven directly.

Finally, aldosterone blockage has also been shown to provide benefit beyond ACEI and ARBs. The largest study to date showing this was the Ephesus trial (Eplerenone Post-AMI Heart Failure Efficacy and Survival Study). This trial showed that the administration of eplerenone, a selective aldosterone blocker, to patients with systolic left ventricular dysfunction (SLVD) post-MI reduced morbidity and mortality.

EPHESUS Results Patients on “standard therapy” in the placebo arm experienced an overall mortality rate of 13.6% at 1 year. The addition of eplerenone resulted in a 15% reduction in all-cause mortality (P=0.008) and a 17% reduction in CV mortality (P=0.005). The combined primary endpoint of CV mortality and CV hospitalization was reduced by 13% (P.002), and the beneficial effects of eplerenone were consistent across patient subgroups. One subgroup that was of particular interest included those patients already receiving extensive neurohormonal blockade with ACE inhibitors and beta-blockers. In this subgroup, the addition of eplerenone further reduced mortality by 27%.

RAAS Inhibitors thus provide mortality and morbidity benefit in individuals with CAD or CAD equivalent.

IV. Cholesterol Modifying Agents

Patients with known CAD or coronary equivalents are at high risk for future adverse cardiovascular events. Cholesterol modifying agent(s), weather they lower the “bad” cholesterol (LDL or Triglycerides) or increase the “good” cholesterol (HDL), have shown reductions in cardiovascular events. One class of such agents is known as statins. But statins are not the only agents that have been show to be effective in cholesterol modification. Agents such as Cholestyramine, Clofibrate, Ezetimibe, Gemfibrozil and niacin (nicotinic acid), have all been shown to modify a patient's cholesterol profile, which provides significant benefit to individuals with CAD or a CAD equivalent.

In terms of individual mechanism of actions, statins acts by competitively inhibiting 3-hydroxy-3-methylglutaryl-coenzyme A (HMG-CoA) reductase, the enzyme that catalyzes the rate-limiting step in cholesterol biosynthesis. Cholestyramine, clofibrate and ezetimibe inhibits absorption of cholesterol at the brush border of the small intestine, leading to a decreased delivery of cholesterol to the liver, reduction of hepatic cholesterol stores and an increased clearance of cholesterol from the blood; decreases total cholesterol, low density lipoprotein (LDL) cholesterol (LDL-C), Apolipoprotein B-100 (ApoB), and triglycerides (TG) while increasing HDL-cholesterol (HDL-C). Gemfibrozil can inhibit lipolysis and decrease subsequent hepatic fatty acid uptake as well as inhibit hepatic secretion of very low-density lipoprotein (VLDL). Together, these actions decrease serum VLDL levels and increases HDL-cholesterol, though the mechanism behind the HDL elevation is currently unknown. Niacin (Nicotinic acid), lowers serum levels of total cholesterol, low-density lipoprotein cholesterol (LDL-C), very low-density lipoprotein (VLDL) and triglycerides. High dose nicotinic acid also increases serum levels of high-density lipoprotein cholesterol (HDL-C) and decreases serum levels of lipoprotein (a) [Lp(a)] and apolipoprotein B-100 (Apo B). The mechanism of the antihyperlipidemic action of nicotinic acid is not well understood. It is thought that this effect is mediated, in part, via decreases in the release of free fatty acids from adipose tissue, thereby decreasing the influx of free fatty acids into the liver, the hepatic reesterification of free fatty acids and the rate of production of hepatic very low-density lipoprotein (VLDL). A decrease in the hepatic production of VLDL reduces the level of circulating VLDL available for conversion to LDL. Another hypothesis holds that nicotinic acid directly inhibits hepatic synthesis or secretion of apolipoprotein B-containing lipoproteins. Still another hypothesis holds that nicotinic acid has the potential to cause a generalized inhibition of synthetic function in the liver. This mechanism may be considered a manifestation of nicotinic acid hepatotoxicity resulting in decreased LDL-cholesterol. However, this liver-damaging hypothesis would not explain the HDL-elevating effect of nicotinic acid. The mechanism by which nicotinic acid elevates HDL is unknown. Nevertheless, high dose nicotinic acid has been found to significantly decrease cardiovascular and cerebrovascular events in those with coronary heart disease. It is thought that this effect is due, in part, to nicotinic acid's antihyperlipidemic activity.

Some particularly preferred examples for useful cholesterol lowering agents in the combination therapy of the present invention are statins. The conversion of 3-hydroxy-methylglutaryl-coenzyme A (HMGCoA) to mevalonate is an early and rate-limiting step in the cholesterol biosynthetic pathway. This step is catalyzed by the enzyme HMOCoA reductase. Statins inhibit HMGCoA reductase from catalyzing this conversion. As such, statins are collectively potent, cholesterol lowering agents. Statins that are suitable for use in the combination therapy and method of the present invention include such compounds as simvastatin, disclosed in U.S. Pat. No. 4,444,784, pravastatin, disclosed in U.S. Pat. No. 4,346,227, cerivastatin, disclosed in U.S. Pat. No. 5,502,199 mevastatin, disclosed in U.S. Pat. No. 3,983,140, velostatin, disclosed in U.S. Pat. No. 4,448,784 and U.S. Pat. No. 4,450,171; fluvastatin, disclosed in U.S. Pat. No. 4,739,073, compactin, disclosed in U.S. Pat. No. 4,804,770; lovastatin, disclosed in U.S. Pat. No. 4,231,938; dalvastatin, disclosed in EP-A 738510, fluindostatin, disclosed in EP-A 363934; atorvastatin, disclosed in U.S. Pat. No. 4,681,893, atorvastatin calcium, disclosed in U.S. Pat. No. 5,273,995; and dihydrocompactin, disclosed in U.S. Pat. No. 4,450,171, all of the above mentioned documents being incorporated expressly herein by reference. Preferred statins include lovastatin, pravastatin, simvastatin, fluvastatin, and atorvastatin.

While there is notable improvement in survival and reduced CV complication with lipid modification, the mechanisms of the benefit are incompletely understood. Regression of atherosclerosis occurs in only a minority of patients after six months of therapy, although one study using magnetic resonance imaging found significant regression of aortic and carotid atherosclerosis after 12 months of therapy with simvastatin. However, the benefit of lipid lowering is seen in as little as six months, before significant regression could occur. Thus, other factors must contribute, which include plaque stabilization, reversal of endothelial dysfunction, and decreased thrombogenicity.

Regardless of the mechanism of benefit, a multitude of trials attest to the benefit of cholesterol modification in individuals with CAD or CAD equivalent. Below are some of the major trials of note.

The Heart Protection Study (HPS) showed a consistent and early benefit of statin therapy in patients with CHD, including those with LDL-C values below 116 mg/dL (3.0 mmol/L), and even below 100 mg/dL (2.6 mmol/L).

Earlier studies of lipid-lowering therapy with medications other than statins showed similar trends toward reductions in cardiovascular events. These included large trials of cholestyramine, clofibrate, and gemfibrozil in primary prevention and a large trial of gemfibrozil in secondary prevention.

The Cholesterol Lowering Atherosclerosis Study (CLAS) examined the hypothesis that a reduction in LDL-cholesterol and elevation in HDL-cholesterol could prevent progression, or cause regression of atherosclerotic lesions at two and four years of follow-up. CLAS included 188 men with previous coronary artery bypass grafts independent of the plasma cholesterol concentration. The patients were randomized to diet plus placebo or diet plus combined therapy with colestipol (30 g per day) and nicotinic acid (3 to 12 g daily). The participants were preselected to ensure tolerance to nicotinic acid and colestipol. The treated group had a 43 percent reduction in LDL-cholesterol and a 37 percent elevation in HDL-cholesterol. The following treatment benefits were noted:
Regression was greater with drug treatment at two years (16.2 versus 2.4 percent) and at four years (18 versus 6 percent).
Progression of old lesions and the development of new lesions were significantly less common in both native and grafted vessels.
Progression was more common in bypass grafts and lesions proximal to grafts than in native arteries.

The benefit of combined nicotinic acid and colestipol therapy was most prominent in patients with baseline plasma cholesterol levels above 240 mg/dL (6.2 mmol/L).

The ARBITER 2 study was a randomized trial which examined the effects of extended-release (ER) niacin 1000 mg daily in 167 patients with known CHD and an HDL-C concentration below 45 mg/dL (1.16 mmol/L) who were already receiving a statin. Patients treated with ER niacin experienced a mean increase in HDL-C of 21 percent from 39 mg/dL (1.0 mmol/L) at baseline to 47 mg/dL (1.22 mmol/L) on treatment (absolute change of 8 mg/dL [0.21 mmol/L]), while patients treated with placebo had no change in HDL-C concentration. Carotid intima-media thickness (CIMT) was assessed by ultrasound. After one year of therapy, mean CIMT increased significantly in the placebo group (0.044 mm, p<0.001), but not in the niacin group (0.014 mm, p=0.23). The difference between these rates approached being statistically significant (p=0.08). Cardiovascular events occurred in 3.8 percent of patients receiving niacin and in 9.6 percent of patients receiving placebo (p=0.20).

One meta-analysis of 17,617 patients in the 4S, CARE, and LIPID trials found that the reduction in the risk of major coronary events, cardiovascular mortality, and all-cause mortality was 30, 27, and 23 percent, respectively. The risk reduction was similar for men and women and for elderly and middle-aged persons.

Similar findings were noted in an analysis of pooled data from three randomized trials of pravastatin involving 19,768 patients (CARE, LIPID, and WOSCOPS). The benefits were similar in younger (<65 years) and older (≧65 years) patients, men and women, smokers and nonsmokers, patients with and without diabetes and hypertension, and across the population ranges of total cholesterol, LDL cholesterol (125 to 212 mg/dL, 3.2 to 5.5 mmol/L), HDL cholesterol, and triglycerides.

Cholesterol modification therefore seems like an integral to improving survival and progression of disease in individuals with CAD.

V. Combination Therapy

Once an individual develops CAD, their risk of death or future adverse events increases substantially. It is evident that each class of agents disclosed previously, namely, anti-platelet agent, Beta blockers, RAAS inhibitor and Cholesterol modifying agent, all confer survival benefit and decrease future cardiovascular complications in patients with CAD and a CAD equivalent.

However, administration of all of these agents separately makes for a very complex and intimidating regimen for the patient. But a medication that would combine each of these classes would confer the benefit of each class and additionally simplify the regimen for the patient. This medication could also improve patient compliance, as compliance has been shown to decrease as the number of medications taken by the patient increases.

By means of combined administration, the effect of one therapy component can be potentated by the other respective component, i.e. the beneficial action of the combination therapy or preparation is more effective than the action of the respective individual components i.e., the components produce a synergistic effect. This combined administration therefore reduces the amount of the respective combination component required for a desired effect, compared with individual administration. The combination therapy and preparation of the present invention accordingly have the advantage that the amounts of compounds to be administered can be significantly reduced and consequently that undesired side effects may be greatly reduced.

To this end, the medication of the present invention combines each comprised of an anti-platelet agent(s), a beta blocker, an inhibitor of the renin angiotensin-aldosterone system (RAAS) and/or a cholesterol modifying agent(s) to form a medication having the benefits of each class of medication combined therein. This “combination therapy” can be used as a pharmaceutical, in mixtures with one another or in the form of suitable pharmaceutical preparations.

The present invention also relates to medications or pharmaceutical preparations and formulations including at least one of each of these classes of medications as active ingredients in addition to customary pharmaceutically innocuous excipients and auxiliaries and their use in the prevention of cardiac events and the production of medicaments thereof. The medication or pharmaceutical preparations normally contain 0.1 to 99 percent by weight, preferably 0.5 to 95 percent by weight of the active ingredients. The pharmaceutical preparations can be prepared in any suitable and well-known manner. To this end, the active ingredients are brought together with one or more solid or liquid pharmaceutical excipients and/or auxiliaries and, if desired, in combination with other pharmaceutical active compounds into a suitable administration form or dose form, which can then be used as a pharmaceutical.

This combination therapy can be administered orally, parenterally, intravenously, rectally or by inhalation, the preferred administration being clinical need. The person skilled in the art is familiar on the basis of his expert knowledge with the auxiliaries which are suitable for the desired pharmaceutical formulation. In addition to solvents, gel-forming agents, suppository bases, tablet auxiliaries and other active compound excipients, it is possible to use, for example, antioxidants, dispersants, emulsifiers, antifoams, flavor corrigents, preservatives, solubilizers, or colorants. In addition to administration as a fixed combination, the combination therapy also includes the simultaneous, separate or sequential administration of the anti-platelet agent(s), the beta-blocker, the inhibitor(s) of RAAS and the cholesterol modifying agents(s) in any of these various forms.

Furthermore, the white ratio of the active compounds in the novel combinations and preparations is preferably in the range of 1-0.01 to 1-100. In addition, the combinations and preparations for the therapy in total may contain 0.5% to 99.5% by weight of these active compounds, and more preferably 1% to 99% by weight of the active compounds. Typically, the individual daily doses for the combinations of active ingredients can range from about 1⅕^{th} of the minimally recommended clinical dosage to the maximum recommended levels for the components when they are given by themselves.

For an oral administration form, in a well known manner, the active compounds are mixed with the additives suitable therefore, such as excipients, stabilizers or inert diluents and are brought by means of the customary methods into the suitable administration forms, such as tablets, coated tablets, hard capsules, aqueous, alcoholic or oily solutions. Inert excipients which can be used are, for example, gum arabic, magnesia, magnesium carbonate, potassium phosphate, lactose, glucose or starch, in particular corn starch. Preparations of the medication of this type can take the form of both dry and moist granules. Possible oily excipients or solvents are, for example, vegetable or animal oils, such as sunflower oil or cod-liver oil.

For subcutaneous or intravenous administration, in a well-known manner, the active compounds are brought into solution, suspension or emulsion, if desired with the substances customary therefore such as solubilizers, emulsifiers or other auxiliaries. Suitable solvents, for example, are: water, physiological saline solution or alcohols, e.g. ethanol, propanol, glycerol, and additionally also sugar solutions such as glucose or mannitol solutions, or alternatively a mixture of the various solvents mentioned.

Pharmaceutical formulations suitable for administration in the form of aerosols or sprays are, for example, solutions, suspensions or emulsions of the active compound of the formula I in a pharmaceutically acceptable solvent, such as, in particular, ethanol or water, or a mixture of such solvents. If required, the formulation can also contain other pharmaceutical auxiliaries such as surfactants, emulsifiers and stabilizers, and also a propellant. Such a preparation customarily contains the active compound in a concentration from approximately 0.1% to 10%, in particular from approximately 0.3% to 3% by weight.

As a result, the combination therapy of the present provides a synergistic effect to each of the components when administered in the combination therapy. The ability of the combination therapy to administer each of these components in a single dosage form also provides great benefits to an individual by effectively reducing the structure required in the therapy.

The dose of active ingredients/compounds to be administered in the medication and the frequency of administration will depend on the potency and duration of action of the compounds used, and additionally on the nature of the indication and on the sex, age, weight and individual responsiveness of the individual to be treated. Dosing regimens will be based on accepted guidelines for efficacy and safety as stated in Prescription Drug Reference (PDR).

Various alternatives are contemplated as being within the scope of the following claims particularly pointing out and distinctly claiming the subject matter regarded as the invention.

I hereby claim:

1. A method for the reduction of either mortality/death or morbidity/complications related to coronary artery disease (CAD) or CAD equivalent comprising the step of administrating to a patient an effective amount of a combined therapy, to treat multiple pathophysiologies that contribute to CAD, which comprises an anti-platelet agent, a beta blocker, an inhibitor of the renin-angiotensin-aldosterone system (RAAS) and/or a cholesterol modifying agent.

2. The method of claim 1 comprising administrating each of the components of the combined therapy simultaneously.

3. The method of claim 2 comprising administrating each of the components of the combined therapy in a single dosage form.

4. The method of claim 3 wherein the dosage form is selected from the group consisting of: an oral dosage form, a parenteral dosage form, an intravenous dosage form, a rectal dosage form and an inhalation dosage form.

5. The method of claim 1 comprising administering each of the components of the combined therapy separately.

6. The method of claim 5 further comprising the steps of:
a) administering a first component of the combined therapy;
b) administering a second component of the combined therapy;
c) administering a third component of the combined therapy; and
d) administering a fourth component of the combined therapy.

7. The method of claim 5 comprising administering each of the components of the combined therapy in separate dosage forms.

8. The method of claim 7 wherein the dosage forms are selected from the group consisting of: an oral dosage form, a parenteral dosage form, an intravenous form, a rectal dosage form and an inhalation dosage form.

9. The method of claim 1 wherein the RAAS inhibitor is selected from the group consisting of an ACE inhibitor, an ARB and/or an aldosterone blocker.

10. The method of claim 9 where the ACE inhibitor is selected from the group consisting of omapatrilat, MDL100240, alacepril, benazepril, captopril, cilazapril, delapril, enalapril, enalaprilat, fosinopril, fosinoprilat, imidapril, lisinopril, perindopril, quinapril, ramipril, ramiprilat, saralasin acetate, temocapril, trandolapril, trandolaprilat, ceranapril, moexipril, quinaprilat, spirapril or a pharmaceutically acceptable derivative thereof.

11. The method of claim 9 where the ARB is selected from the group consisting of saralasin acetate, candesartan cilexetil, valsartan, candesartan, losartan potassium, eprosartan, irbesartan, tasosartan, telmisartan or a pharmaceutically acceptable derivative thereof.

12. The method of claim 9 where the aldosterone blocker is selected from the group consisting of spironolactone, eplerenone or a pharmaceutically acceptable derivative thereof.

13. The method of claim 1 wherein the cholesterol-modifying agent is selected from the group consisting of an HMG-CoA synthase inhibitor, a squalene epoxidase inhibitor, a squalene synthetase inhibitor, an ACAT inhibitor, probucol, niacin, a fibrate, a cholesterol absorption inhibitor, a bile acid sequestrant, an LDL receptor inducer, an HDL modifier or a pharmaceutically acceptable derivative thereof.

14. The method of claim 1 wherein the antiplatelet agent is selected from the group consisting of aspirin, dipyridamole, clopidogrel, ticlopidine, glycoprotein IIb/IIIa inhibitors, cyclooxygenase inhibitor, ADP receptor blockers or a pharmaceutically acceptable derivative thereof.

15. The method of claim 1 wherein a beta blocker is selected from the group consisting of sotalol, timolol, esmolol, carteolol, carvedilol, nadolol, propranolol, propranolol, betaxolol, penbutolol, metoprolol, labetalol, acebutolol, atenolol, metoprolol, pindolol, bisoprolol or a pharmaceutically acceptable derivative thereof.

16. The method of claim 1 wherein the combined therapy contains 0.5 to 95% by weight of the components of the combined therapy.

17. A medication for use in the reduction of either the mortality/death or morbidity/complications related to coronary artery disease (CAD) or CAD equivalent comprising an effective amount of a combined therapy, to treat multiple pathophysiologies that contribute to CAD, including an anti-platelet agent(s), a beta blocker, an inhibitor(s) of the renin-angiotensin-aldosterone system (RAAS) and a cholesterol modifying agent.

18. A medication of claim 17 wherein a dosage form for the medication is selected from the group consisting of: an oral dosage form, a parenteral dosage form, an Intravenous dosage form, a rectal dosage form, and an Inhalation dosage form.

19. The medication of claim 17 wherein the RAAS inhibitor is selected from the group consisting of an ACE Inhibitor, an ARB and/or an aldosterone blocker.

20. The medication of claim 19 where the ACE inhibitor is selected from the group consisting of omapatrilat, MDL100240, alacepril, benazepril, captopril, cilazapril, delapril, enalapril, enalaprilat, fosinopril, fosinoprilat, imidapril, lisinopril, perindopril, quinapril, ramipril, ramiprilat, saralasin acetate, temocapril, trandolapril, trandolaprilat, ceranapril, moexipril, quinaprilat, spirapril or a pharmaceutically acceptable derivative thereof.

21. The medication of claim 19 where the ARB is selected from the group consisting of saralasin acetate, candesartan cilexetil, valsartan, candesartan, losartan potassium, eprosartan, irbesartan, tasosartan, telmisartan or a pharmaceutically acceptable derivative thereof.

22. The medication of claim 19 where the aldosterone blocker is selected from the group consisting of spironolactone, eplerenone or a pharmaceutically acceptable derivative thereof.

23. The medication of claim 17 wherein the cholesterol-modifying agent is selected from the group consisting of an HMG-CoA synthase inhibitor, a squalene epoxidase inhibitor, a squalene synthetase inhibitor, an ACAT inhibitor, probucol, niacin, a fibrate, a cholesterol absorption inhibitor, a bile acid sequestrant, an LDL receptor inducer, an HDL modifier or a pharmaceutically acceptable derivative thereof.

24. The medication of claim 17 wherein the antiplatelet agent is selected from the group consisting of aspirin, dipyridamole, clopidogrel, ticlopidine, glycoprotein IIb/IIIa inhibitors, an cyclooxygenase inhibitor, ADP receptor blockers or a pharmaceutically acceptable derivative thereof.

25. The medication of claim 17 wherein a beta blocker is selected from the group consisting of sotalol, timolol, esmolol, carteolol, carvedilol, nadolol, propranolol, propranolol, betaxolol, penbutolol, metoprolol, labetalol, acebutolol, atenolol, metoprolol, pindolol, bisoprolol, or a pharmaceutically acceptable derivative thereof.

26. The medication of claim 17 wherein the combined therapy contains 0.5 to 95% by weight of the components of the combined therapy.

27. A method for the reduction of either the mortality/death or morbidity/complications related to coronary artery disease or CAD equivalent comprising the step of administrating to a patient an effective amount of combined therapy which includes two or more of: an antiplatlet agent(s), beta blocker, inhibitor(s) of the renin-angiotensin-aldosterone system and/or cholesterol modifying agent(s) together with an additional medication useful in the treatment and prevention of coronary artery disease.

28. A medication for assisting a patient with coronary artery disease (CAD) or CAD equivalent in preventing the onset of future or recurrence of cardiovascular events such as stroke, congestive heart failure, cardiovascular death, myocardial infarction, worsening of angina, and cardiac arrest, the medication comprising the step of administrating to a patient an effective amount of combined therapy which includes two or more of: an antiplatlet agent(s), beta blocker, inhibitor(s) of the renin-angiotensin-aldosterone system and/or cholesterol modifying agent(s) together with an additional medication useful in the treatment and prevention of coronary artery disease.

29. A medication for assisting a patient in preventing need for revascularisation procedures, such as coronary artery bypass graft surgery (CABG), Percutaneous Coronary Angioplasty (PTCA), peripheral angioplasty surgery, amputation, and carotid endarterectomy, the medication comprising the step of administrating to a patient an effective amount of combined therapy which includes two or more of: an antiplatlet agent(s), beta blocker, inhibitor(s) of the renin-angiotensin-aldosterone system and/or cholesterol modifying agent(s) together with an additional medication useful in the treatment and prevention of coronary artery disease

30. A combination therapy medication that retards the progression of, or reverse CAD, the medication comprising an effective amount of two or more of: an antiplatlet agent(s), beta blocker, inhibitor(s) of the renin-angiotensin-aldosterone system and/or cholesterol modifying agent(s) together with an additional medication useful in the treatment and prevention of coronary artery disease.

31. A method for halting or slowing the progression of atherosclerotic disease comprising the administration of a therapeutically effective amount of the medication of claim 30, to a person with atherosclerotic disease.

32. A method for preventing or reducing the risk of occurrence, or recurrence where the potential exists, of an atherosclerotic disease event comprising the administration of a therapeutically effective amount of the medication of claim 30, to a person at risk of having an atherosclerotic disease event.

33. A combination therapy medication that assists a patient in preventing diabetic complications such as overt nephropathy or new microalbuminuria, the medication comprising an effective amount of two or more of: an antiplatlet agent(s), beta blocker, inhibitor(s) of the renin-angiotensin-aldosterone system and/or cholesterol modifying agent(s) together with an additional medication useful in the treatment and prevention of coronary artery disease
